(19) United States
(12) Patent Application Publication
(10) Pub. No.: US 2025/0143549 A1
(43) Pub. Date: May 8, 2025

(54) DEVICES AND SYSTEMS FOR A FLEXIBLE ELONGATE MEMBER INSERTABLE INTO A BODY

(71) Applicant: BOSTON SCIENTIFIC SCIMED, INC., Maple Grove, MN (US)

(72) Inventors: Allison Kumarasena, Wellesley, MA (US); Austin Grant Johnson, Worcester, MA (US)

(73) Assignee: BOSTON SCIENTIFIC SCIMED, INC., Maple Grove, MN (US)

(21) Appl. No.: 18/935,084

(22) Filed: Nov. 1, 2024

Related U.S. Application Data
(60) Provisional application No. 63/595,547, filed on Nov. 2, 2023.

(51) Int. Cl. A61B 1/00; A61B 1/005

(52) U.S. Cl. CPC A61B 1/00137; A61B 1/005

(57) ABSTRACT

Devices, systems, and methods of or for use with an elongate shaft of a medical scope. The device may be or may include a cap for the elongate shaft. The cap may include a proximal end, a distal end, a lumen extending between the proximal end and the distal end, and a protrusion located between the proximal end and the distal end. The proximal end of the cap may be configured to couple with the elongate shaft. The distal end of the cap may define a first diameter. The protrusion may define a second diameter that is smaller than the first diameter. The protrusion may be configured to engage tissue received in the lumen through the distal end.

### CROSS-REFERENCE TO RELATED APPLICATIONS

This application claims the benefit of priority under 35 U.S.C. § 119 to U.S. Provisional Application No. 63/595,547, filed Nov. 2, 2023, the entire disclosure of which is hereby incorporated by reference herein for all purposes.

### TECHNICAL FIELD

This disclosure relates generally to cap devices, systems, and methods, and particularly to caps for coupling with flexible elongate members insertable into a body.

### BACKGROUND

A wide variety of intracorporeal medical devices and systems have been developed for medical use, for example, for transluminal procedures. Some of these devices and systems include guidewires, catheters, catheter systems, transluminal instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In a first example, a cap for a flexible elongate member insertable into a body may comprise a proximal end configured to couple with the flexible elongate member, a distal end defining a first inner diameter, a lumen extending between the proximal end and the distal end, and a protrusion located between the proximal end and the distal end and defining a second inner diameter that is smaller than the first inner diameter, wherein the protrusion may be configured to engage tissue received in the lumen through the distal end.

Alternatively or additionally to any of the examples above, the protrusion may be configured to be spaced distally apart from a distal end of the flexible elongate member when the proximal end is coupled with the flexible elongate member.

Alternatively or additionally to any of the examples above, the protrusion may extend circumferentially around the lumen.

Alternatively or additionally to any of the examples above, the protrusion may define a non-circular end of the lumen.

Alternatively or additionally to any of the examples above, the protrusion may define a circular end of the lumen.

Alternatively or additionally to any of the examples above, the distal end may be made from a substantially transparent material.

In a further example, a device may comprise a medical scope having a proximal end and a distal end and a cap configured to couple with the distal end of the medical scope, the cap comprising a proximal end, a distal end, and a protrusion having a distal surface configured to engage tissue received through an opening at the distal end.

Alternatively or additionally to any of the examples above, the protrusion may be spaced distally apart from the distal end of the medical scope when the cap is coupled with the distal end of the medical scope.

Alternatively or additionally to any of the examples above, an inner surface of the cap may define a lumen and the lumen has a first diameter at a distal end of the lumen that is greater than a second diameter at the protrusion.

Alternatively or additionally to any of the examples above, the protrusion may extend circumferentially around the lumen.

Alternatively or additionally to any of the examples above, the protrusion may define a non-circular portion of the lumen.

Alternatively or additionally to any of the examples above, the protrusion may define a circular portion of the lumen.

Alternatively or additionally to any of the examples above, the medical scope may have a working channel and an aspiration channel.

Alternatively or additionally to any of the examples above, the distal surface of the protrusion and the distal end of the cap may be configured to contact tissue when a negative pressure is applied to the aspiration channel.

Alternatively or additionally to any of the examples above, the device may further comprise a cutting device extending through the working channel, wherein the cutting device may be configured to cut through a surface of the tissue at a location between two portions of the distal surface engaging the tissue.

In a further example, a method of creating an incision through a first tissue to a second tissue may comprise engaging a distal end of a cap and a protrusion of the cap with the first tissue, wherein the protrusion may be proximally offset from the distal end of the cap, and cutting through the first tissue to the second tissue at an axial location along the cap proximate an axial location of the protrusion.

Alternatively or additionally to any of the examples above, the method may further comprise applying a negative pressure to the first tissue through a lumen extending through the cap.

Alternatively or additionally to any of the examples above, engaging the distal end of the cap and the protrusion of the cap with first tissue may comprise engaging the distal end of the cap with the first tissue and applying a negative pressure to the first tissue to draw the first tissue into the cap to engage the protrusion.

Alternatively or additionally to any of the examples above, a proximal end of the cap may be coupled to a distal end of a medical scope and the medical scope may have a working channel.

Alternatively or additionally to any of the examples above, the method may further comprise extending a cutting device through the working channel to a location proximate the axial location of the protrusion.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative medical scope;

FIG. 2 depicts a schematic view of a distal end of an illustrative medical scope shaft with a cap coupled therewith and proximate tissue;

FIG. 3. depicts a schematic view of a distal end of an illustrative medical scope shaft with a cap coupled therewith and proximate tissue;

FIG. 4 depicts a schematic perspective view of a distal end of a medical scope with an illustrative cap coupled therewith;

FIG. 5 depicts a schematic perspective view of the distal end of the medical scope with the illustrative cap depicted in FIG. 4 coupled therewith and proximate tissue;

FIG. 6 depicts a schematic cross-section view of an illustrative cap;

FIG. 7 depicts a schematic cross-section view of an illustrative cap;

FIG. 8 depicts a schematic view through an illustrative cap positioned proximate tissue;

FIG. 9 depicts a schematic view through an illustrative cap positioned proximate tissue; and

FIGS. 10A-10D depict a schematic technique of using a medical scope with an illustrative cap coupled with a distal end of the medical scope.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in transluminal medical procedures. However, it should be noted that reference to this particular procedure is provided only for convenience and not intended to limit the disclosure. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “a configuration”, “some configurations”, “other configurations”, etc., indicate that the configuration(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with a configuration, the particular feature, structure, or characteristic may be implemented in connection with other configurations, whether or not explicitly described, unless clearly stated to the contrary. That is, the various individual elements described below, even if not explicitly shown in a particular combination, are nevertheless contemplated as being combinable or arrangeable with each other to form other additional configurations or to complement and/or enrich the described configuration(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

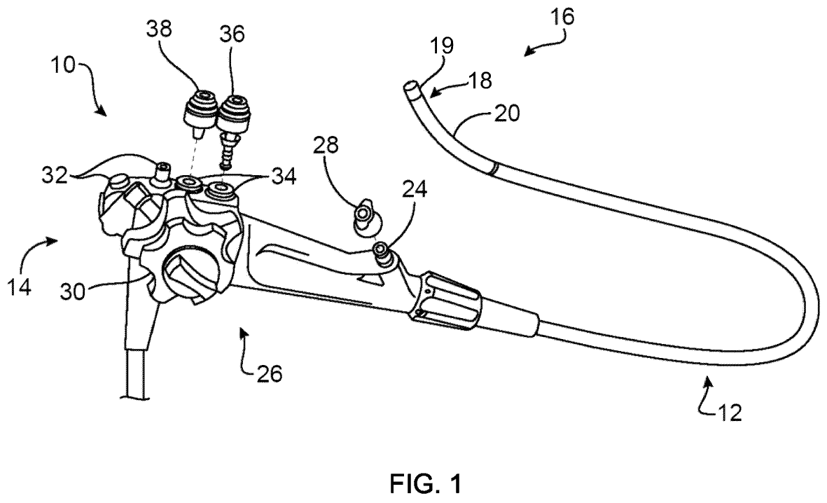

With reference to FIG. 1, an illustrative medical scope 10, such as an endoscope, is depicted in a schematic perspective view. The medical scope 10 may include an elongate tube or shaft 12 (e.g., a flexible elongate shaft or member) that is configured to be inserted into a subject (e.g., a patient).

The medical scope 10 may include a proximal portion 14 and a distal portion 16. The distal portion 16 of the medical scope 10 may house an imager (e.g., CCD or CMOS imager) (not shown). In some cases, a light source of a medical scope system including the medical scope 10 may feed illumination light to a distal portion 16 of the medical scope 10.

The elongate shaft 12 may include a distal end 18 (e.g., a distal end or end unit adapted to be inserted into a body cavity of a patient) provided at and/or along the distal portion 16 of the shaft 12 and a flexible bending portion 20 proximal of the distal end 18, but other suitable configurations are contemplated. The flexible bending portion 20 may include an articulation joint (not shown) to assist with steering the distal end 18, but this is not required.

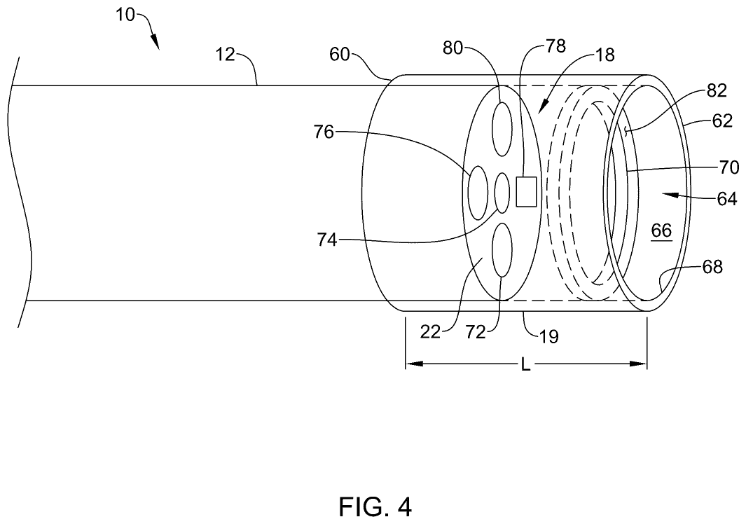

The distal end 18 of the medical scope 10 may include an end face (e.g., an end face 22 as depicted in FIG. 4). In some cases, the end face may have a gas/lens wash nozzle for supplying gas to insufflate the interior of the patient at the treatment area and for supplying water to wash a lens covering the imager, an irrigation opening that may supply irrigation fluid to the treatment area (e.g., a target site) of the patient, one or more illumination windows that convey illumination light to the treatment area, an opening to an aspiration channel extending along the shaft 12, and/or an opening to a working channel extending along the shaft 12 for passing tools to the treatment area. In some examples, the working channel may extend along the shaft 12 to a proximal channel opening 24 positioned at or along an operating handle 26 (e.g., a proximal handle) of the medical scope 10. A biopsy valve 28 may be utilized to seal the channel opening 24 against unwanted fluid egress. Further, a cap 19 may be located at or on the distal end 18 of the medical scope 10 and may extending distally from the distal end 18.

The operating handle 26 may be provided with one or more knobs 30 for providing steering, such as remote 4-way steering, of the distal end 18 via wires connected to the articulation joint in the flexible bending portion 20 (e.g., one knob may control up-down steering and another knob may control left-right steering). A plurality of video switches 32 for remotely operating a video processing unit may be arranged on the handle 26 at the proximal portion 14 of the medical scope 10.

The handle 26 may be provided with dual valve locations 34. One of the valve locations 34 may have or receive a gas/water valve 36 for operating an insufflating gas and lens water feed operation. A gas supply line and a lens wash supply line may run distally from the gas/water valve 36 along the shaft 12 and converge at the distal end 18 proximal to the gas/wash nozzle, but other suitable configurations are contemplated.

The other valve location 34 may have or receive a suction valve 38 for operating a suction operation. A suction supply line may run distally from the suction valve 38 along the shaft 12 to a junction point in fluid communication with the working channel of the medical scope 10.

Although an illustrative medical scope 10 is described with respect to FIG. 1, other suitable configurations of the medical scope 10 are contemplated and one or more other suitable delivery devices (e.g., scopes, tubes, shafts, etc.) may be utilized with the cap configurations or concepts discussed herein. In some examples, an illustrative configuration of a delivery device may include the elongate shaft 12 and the handle 26, along with other suitable components for using the delivery device (e.g., the medical scope 10 and/or one or more other suitable delivery devices) within a subject (e.g., within the patient).

The medical scope 10 and/or other delivery devices may be configured to perform and/or facilitate performing “third space” procedures and/or procedures in other transluminal spaces of the subject that are difficult to access with the distal end 18 of the medical scope 10. The “third space” of a subject may be a space inside a wall of a gastrointestinal (GI) tract of the subject, such as in or between a mucosal layer of tissue and a submucosal layer of tissue. Example third space procedures include, but are not limited to, per oral endoscopic myotomy (POEM), endoscopic submucosal dissection (ESD), Zenker's diverticulotomy (ZPOEM), endoscopic gastric pyloromyotomy (GPOEM), submucosal tunneling endoscopic resection (STER), and tunneling recanalization of the esophagus (POETRE), among others. These third space procedures and/or other suitable procedures may be used to treat esophageal sphincters, pyloric sphincters, subepithelial tumors, and/or other anatomical structures.

Some third space procedures (e.g., POEM, STER, POETRE) may include tunneling to or into the sub-mucosal tissue layer of the subject and performing one or more additional procedural steps. To create the tunnel, an incision may be made that extends from the mucosal layer of tissue to or into the submucosal layer of tissue such that a diameter of a scope may fit through the opening.

The creating of the incision from the mucosal tissue layer to or into the submucosal tissue layer can be challenging and time intensive. In some examples, an angle of approach of the medical scope 10 and/or other delivery device and the cutting tool (e.g., an electrosurgical knife and/or other suitable cutting tool) may affect the difficulty of making a proper incision from the mucosal tissue layer to or into the submucosal tissue layer.

A cover or cap 19 connected to and/or coupled with the distal end of the elongate shaft 12 of the medical scope 10 and/or other delivery device may be utilized for third space procedures in order to maintain visualization of a target and to facilitate creating a tunnel to and/or into the submucosal layer of tissue. In some examples, the cover or cap 19 may be clear or transparent (e.g., the cover or cap 19 may be made from any suitable clear or transparent biocompatible material) and create a space distal of the distal end 18 of the medical scope 10 that is isolated from body fluids and/or non-targeted tissue.

The cover or cap 19 may be coupled with the distal end of the elongate shaft 12 in any suitable manner including, but not limited to, via a threaded connection, a luer lock connection, a friction fit, a keyed connection, a ball-detent connection, and/or other suitable type of connection. In some cases, the coupling between the cap 19 and the elongate shaft 12 may be configured to prevent the cap 19 from adjusting proximally, distally, and/or rotationally relative to the elongate shaft 12 during a procedure.

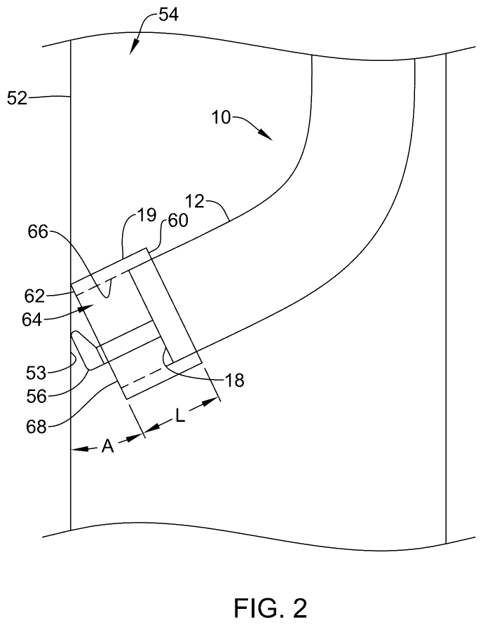

The cap 19 may overlap with the distal end of the elongate shaft 12, as depicted for example in FIG. 2. In some examples, the cap 19 may over lap with the distal end of the elongate shaft 12 any suitable distance, which may include, but is not limited to, a distance in a range of about one (1) millimeter (mm) to about twenty-five (25) mm.

The cap 19, when utilized, may have a length L that is rigid, which makes it difficult for a distal end 62 of the cap 19 to be flush or perpendicular to a target surface 53 (e.g., a target surface of a tissue wall 52 in a GI tract 54 and/or other suitable target site). FIG. 2 depicts an illustrative example of a non-zero or non-parallel angle A between the medical scope 10 (e.g. the cap 19, a cutting tool 56 (e.g., an electrosurgical knife and/or other suitable cutting tool), the distal end 18, etc., which all may or may not have a surface parallel to one another) and the tissue wall 52. The non-zero angle A or non-parallel relationship between the tissue wall 52 and one or more components of the medical scope 10, may increase a difficulty of creating a straight and/or level incision to or into the submucosal layer of tissue with the cutting tool 56. The angle A between the medical scope 10 and the tissue wall 52 may increase as a diameter of the body lumen narrows relative to a diameter or size of the medical scope 10 and/or the cap 19 (reducing the ability of the distal end 18 of the medical scope 10 to bend to a position closer to parallel to the tissue wall 52). An example of a relatively narrow body lumen is an esophagus of a subject, but use of devices disclosed herein in other narrow body lumens is contemplated.

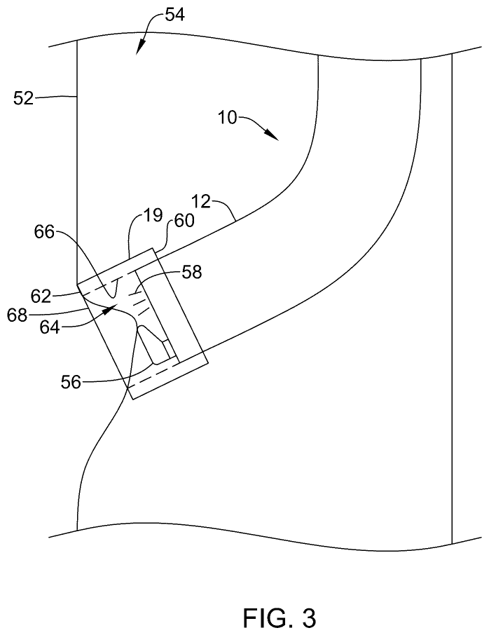

The cap 19 may have a proximal end 60, a distal end 62, and a lumen 64 (e.g., a working channel or other suitable lumen) extending between or along the proximal end 60 and the distal end 62. In some examples, the lumen 64 may be defined by an inner surface 66 (e.g., shown in broken lines in FIG. 2) and thus, a diameter defined by the inner surface 66. The diameter of the lumen 64 may be constant or may vary along the length L of the cap 19. The distal end 62 of the cap 19 may include an opening 68 configured to permit tissue (e.g., soft tissue of the tissue wall 52) to be drawn into the lumen 64 of the cap 19 using suction or negative pressure (e.g., as depicted in FIG. 3, which is discussed below).

The cap 19 may have any suitable shape configured to couple with the distal end 18 of the medical scope 10, facilitate traversing body lumens, and/or create space for users to view tissue by spacing the tissue from the distal end 18 of the medical scope 10. In some examples, the cap 19 may have a cylindrical shape, but other suitable shapes are contemplated. Further, the shape of the cap 19 may or may not be circumferentially constant along the length L of the cap 19.

As depicted, for example, in FIG. 3, suction or negative pressure 58 at the distal end 18 and/or the cap 19 (e.g., represented by lines extending out of the distal end 18) may be utilized to reduce an angle between the medical scope 10 and/or the cutting tool 56 and the target surface 53 at the tissue wall 52. In the example of utilizing suction or negative pressure 58, the tissue wall 52 may be suctioned into (as depicted in FIG. 3) or toward the cap 19 to reduce an angle between the cutting tool 56 and at least a portion of the target surface 53. The suction, however, may result in inconsistent or unpredictable acquisition of the tissue wall 52 in the lumen 64 of the cap 19 due to doming of the tissue wall 52 such that sufficient control of incision location and/or depth in the tissue wall 52 at the target surface 53 by the cutting tool 56 may not occur.

Suction or negative pressure 58 may be applied to the tissue wall 52 in any suitable manner. In some examples, a user may not be able to control, or at least accurately control, an amount of negative pressure that is applied to the tissue wall 52 (e.g., the negative pressure may not be accurately adjustable). In some cases, the negative pressure may be predetermined and may not be adjustable other than to be turned on or off. In other examples, a user may be able to accurately adjust or control an amount of negative pressure that is applied to the tissue wall 52.

To facilitate creating a target surface 53 at which the cutting tool 56 may be used to make a predictable incision (e.g., an incision at a predictable or desired location, of a predictable or desired size, and/or of a predictable and/or desirable depth), one or more protrusions may be formed in a wall of the cap 19 and defined by the inner surface 66. In some examples, the protrusion may be configured to contact tissue drawn into the lumen 64 of the cap 19 to flatten out a surface of that tissue and create a predictably configured target surface that is parallel to or substantially parallel to a cutting surface of the cutting tool 56. Even when the distal end 62 of the cap 19 is non-parallel to the tissue wall 52, the protrusion in the cap 19 may contact the drawn-in tissue and create a surface of the tissue that is parallel to or substantially parallel to the cutting tool 56 so as to facilitate creating a consistent or predictable incision in the tissue that may allow for making an initial tunnel through the tissue wall 52 for third space procedures and/or other suitable procedures.

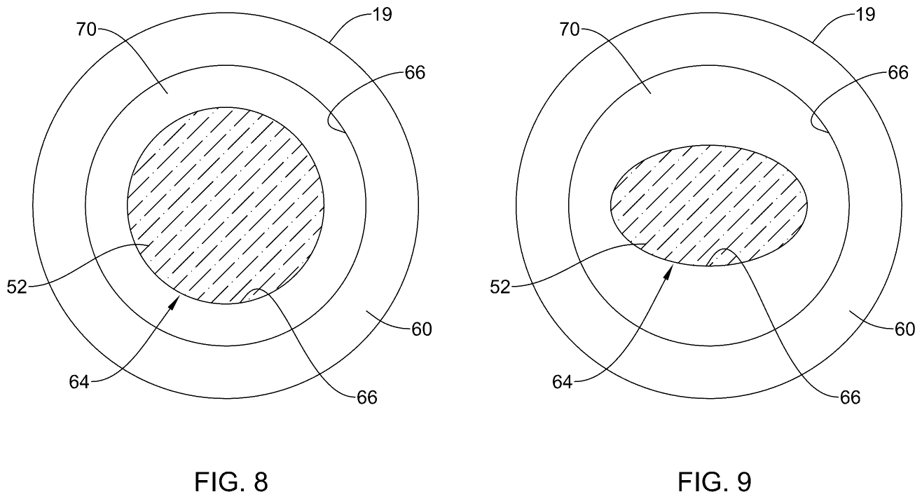

FIG. 4 schematically depicts an illustrative configuration of the cap 19 coupled with the elongate shaft 12 of the medical scope 10, where the cap 19 includes a protrusion 70 (e.g., a radially inwardly-directed protrusion, such as shown in broken lines when viewed through the cap 19, as in FIG. 4 and in solid lines when viewed through the opening 68 at the distal end 62 of the cap 19, as in FIG. 8). Similar to as discussed above, the cap 19 may be coupled with the distal end 18 of the elongate shaft 12. In some examples, the end face 22 of the elongate shaft 12 may face the lumen 64 of the cap 19. The end face 22 may include one or more openings. In some examples, the openings in the end face 22 may include, but are not limited to, an opening for one or more working channels 72, an opening for one or more suction/aspiration channels 74, an opening for one or more irrigation channels 76 (e.g., for delivering water, liquid, gas, to the lumen 64 of the cap 19), an opening for one or more imaging or viewing channels 78, an opening for one or more light guide channels 80, and/or one or more other suitable channels.

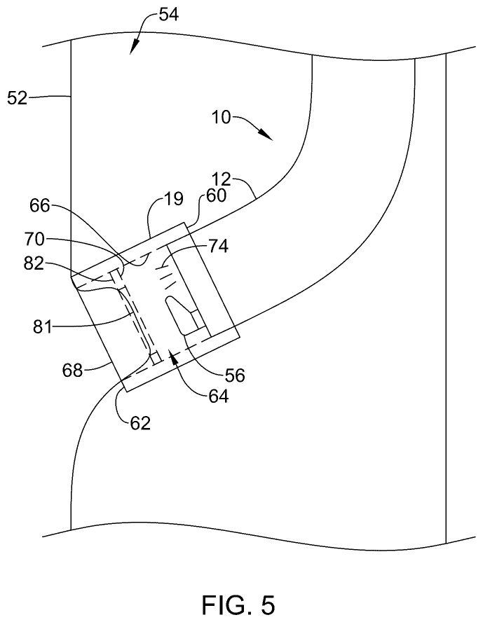

When tissue is drawn into the cap 19, the tissue may abut the distal end 62 of the cap 19 and then further abut the protrusion 70 (e.g., further abut a distal surface 82 of the protrusion 70 and/or other suitable surface of the protrusion 70) to limit a distance the tissue is drawn into the cap 19 and create a flat target surface 81 in which an incision may be formed, as depicted, for example in FIG. 5. In some examples, the protrusion 70 may include a distal surface 82 that is configured to abut the tissue drawn into the cap 19 through the opening 68 and affect a shape of the target surface 81.

The cap 19 may include one or more protrusions 70. For example, the cap 19 may include a single protrusion 70 (e.g., as depicted in FIG. 4, such as substantially continuous, circumferentially-extending protrusion), two protrusions 70, three protrusions 70, five protrusions 70, and/or other suitable number of protrusions 70.

The protrusion(s) 70 may be at any suitable location(s) in the lumen 64 of the cap 19. For example, the protrusion(s) 70 may be inset proximally from the distal end 62 of the cap 19 and/or inset distally from the proximal end 60 of the cap 19. In some examples, proximally insetting the protrusion 70 from the distal end 62 may facilitate maintaining a desired negative pressure at the opening 68 of the cap 19, as reducing the diameter of the lumen 64 at the distal end 62 may cause a higher pressure at the distal end than desired which may frustrate creating a desired surface for forming an incision. In some examples, distally insetting the protrusion 70 from the proximal end 60 may facilitate providing suitable space for instruments, imaging, lighting, suction, and/or other features at and/or within the cap 19. In one example configuration of the cap 19, as depicted in FIG. 4, the protrusion 70 may be positioned at a location in the lumen 64 of the cap 19 that is inset proximally from the distal end 62 and inset distally from the end face 22 of the elongate shaft 12 when the cap 19 is coupled with the elongate shaft 12, but other suitable configurations are contemplated. When multiple protrusions 70 are utilized, the protrusions 70 may all be located at a same longitudinal or axial location along the lumen 64 of the cap 19 or one or more of the protrusions 70 may be located at a different longitudinal or axial location along the lumen 64 of the cap 19 than one or more other protrusions 70.

When inset, the protrusion(s) 70 may be inset from one or both of the proximal end 60 and the distal end 62 of the cap 19 any desired or suitable distance. In some examples, the protrusion 70 be inset or spaced apart proximally from the distal end 62 of the cap 19 a suitable distance to draw a sufficient amount of tissue into the cap 19 to engage the protrusion 70 and create a desire surface through which an incision may be formed. Example suitable distances for insetting the protrusion(s) 70 from the distal end include, but are not limited to, distances in a range of about one (1) mm to about twenty five (25) mm. Additionally or alternatively, the protrusion 70 may be spaced apart or inset distally a suitable distance from the proximal end 60 of the cap 19 and/or a distal end of the elongate shaft 12 (e.g., the end face 22), which may include, but are not limited to, the protrusion 70 being inset distally from the end face 22 of the elongate shaft a distance in a range of about four (4) mm to about fifteen (15) mm, inset distally from the proximal end 60 of the cap 19 a distance in a range of about five (5) mm to about forty (40) mm, and/or inset distally one or more other suitable distances.

The protrusion(s) 70 may extend around the lumen 64 any suitable distance. In some examples, a single protrusion 70 may extend circumferentially around the lumen 64 (e.g., extend an entirety of an inner circumference of the lumen 64). Alternatively, in some examples, one or more protrusions 70 may extend around the lumen 64 less than an entire circumference around the lumen 64. When multiple protrusions 70 are utilized, the protrusions may be equally or unequally spaced apart around the circumference of the lumen 64 in some examples. Further, in some examples, when multiple protrusions 70 are utilized and are located at different longitudinal or axial locations along the cap 19, the protrusions 70 may or may not overlap in a circumferential direction.

The protrusion 70 may be defined by the inner surface 66 of the cap 19, may define the lumen 64 of the cap 19, and/or may have any suitable shape. In some examples, the shape of the protrusion may be, but is not limited to, a shelf shape, a ridge shape, a rectangular cross-sectional shape, a rounded cross-sectional shape, a triangular cross-sectional shape, an annular shape, a circular shape, a rectangular shape, an oval shape, a triangular shape, and/or other suitable shape. As discussed, the shape of the protrusion 70 may form the distal surface 82 (e.g., a surface facing distally or toward the opening 68 at the distal end 62 of the cap 19). In one example, the cap 19 may include a single protrusion 70 extending circumferentially around the lumen 64, where the single protrusion 70 may have a ridge or shelf configuration with a rectangular cross-section, but other suitable configurations are contemplated.

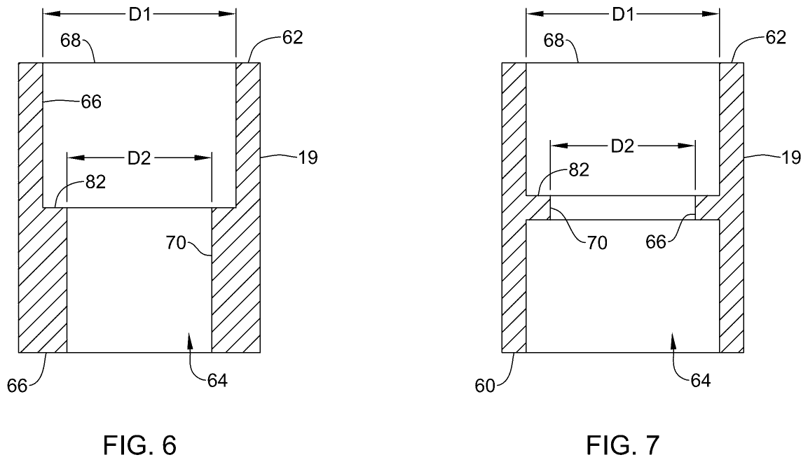

FIGS. 6 and 7 depict schematic cross-section views of illustrative configurations of the cap 19. As depicted in FIGS. 6 and 7, lumen 64 at or proximate the opening 68 at the distal end 62 may have a first diameter D1 (e.g., a first inner diameter) and the lumen 64 at or proximate the distal surface 82 of the protrusion 70 may have a second diameter D2 (e.g., a second inner diameter) that is less than (e.g., smaller than) the first diameter D1 (e.g., the first diameter D1 may be greater than the second diameter D2). Such a configuration may facilitate allowing a desirable amount of tissue into the lumen 64 of the cap 19 (e.g., due to pressure at the distal end 62 and/or the space at the opening 68) and engaging the received tissue with or proximate the distal surface 82 to create a flat or otherwise desirable surface for an incision.

FIG. 6 schematically depicts the protrusion 70 inset proximally from the distal end 62 of the cap 19 and extending to the proximal end 60 of the cap 19. The protrusion 70 extending to the proximal end 60 of the cap 19 may be configured for any suitable purpose. In some examples, the protrusion 70 may extend to the proximal end 60 to facilitate coupling with the distal end 18 of the elongate shaft 12. In one example, the protrusion 70 at the proximal end 60 may be keyed to mate with the elongate shaft 12, such that cap 19 may only couple with the elongate shaft 12 when in a desired position (e.g., angular position, rotational position, axial position, etc.) relative to the elongate shaft 12. Further, when configured to mate with the elongate shaft 12, a proximal surface or other suitable surface of the protrusion 70 may be configured to engage a surface (e.g., the end face 22, a distally facing surface, and/or other suitable surface) of the elongate shaft 12 to prevent proximal movement of the cap 19 relative to the elongate shaft 12 (e.g., due to suction forces at the cap and/or other suitable forces). The protrusion 70 at the proximal end 60 of the cap 19, at the distal surface 82, and at one or more locations between the proximal end 60 of the cap 19 and the distal surface 82 may all have a same shape and/or configuration or one or more locations may have a different shape or configuration than one or more other locations along the protrusion 70.

FIG. 7 schematically depicts the protrusion 70 inset proximally from the distal end 62 of the cap 19 and inset distally from the proximal end 60 of the cap 19. The protrusion 70 spaced apart or inset distally from the proximal end 60 of the cap 19 may facilitate coupling the cap 19 with distal end 18 of the elongate shaft 12 and/or allow additional space, relative to the configuration of the cap 19 depicted in FIG. 6, for the operating of devices or components of the medical scope 10 extending through or along lumens of the elongate shaft 12 and/or for performing functions at or within the cap 19 (e.g., cutting tissue, capturing images, applying a negative pressure to the lumen 64, applying fluid to the lumen 64, etc.)

Further, the cap 19 and/or the elongate shaft 12 may include a stop component that is configured to engage the other of the cap 19 and/or the elongate shaft 12 to prevent longitudinal movement of the cap 19 in the proximal direction relative to the elongate shaft 12 (e.g., during suction and/or at other times). In some examples, the stop may be incorporated into the protrusion 70 (e.g., the stop component may be a proximal facing surface of the protrusion 70 when the protrusion extends to the proximal end 60 of the cap 19), but other suitable configurations are contemplated. In some examples, when the protrusion 70 is inset distally from the proximal end 60 of the cap 19, the stop component may be spaced proximally from the protrusion 70 and may have a surface configured to engage the elongate shaft 12. The stop component may be one or more elements extending circumferentially around or less than circumferentially around an inner circumference of the cap 19 and/or an outer circumference of the elongate shaft 12.

FIGS. 8 and 9 depict schematic views through an axial end of the cap 19, along the lumen 64 from the proximal end 60, wherein the protrusion 70 defines a portion of the lumen 64. The protrusion 70 may define a size and/or shape of the lumen 64 in any suitable manner. In some examples, the protrusion 70 may define the inner surface 66 forming a circular cross-section of the lumen 64, as depicted in FIG. 8. In some examples, the protrusion 70 may define the inner surface 66 forming non-circular cross-section (e.g., oval, elliptical, etc.) of the lumen 64, as depicted in FIG. 9. Other suitable configurations of the protrusion 70 are contemplated.

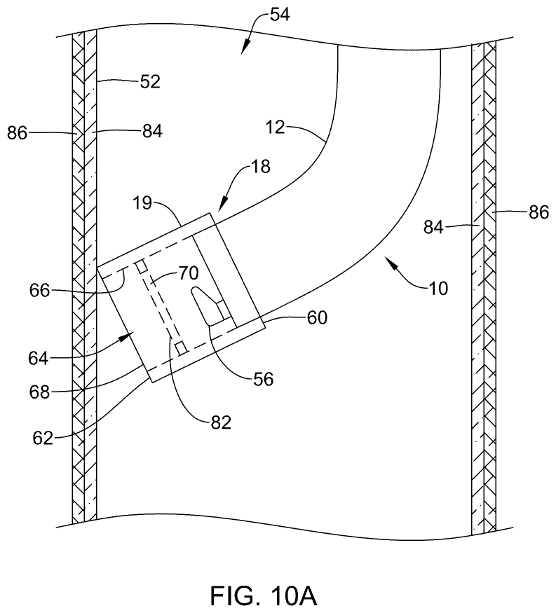

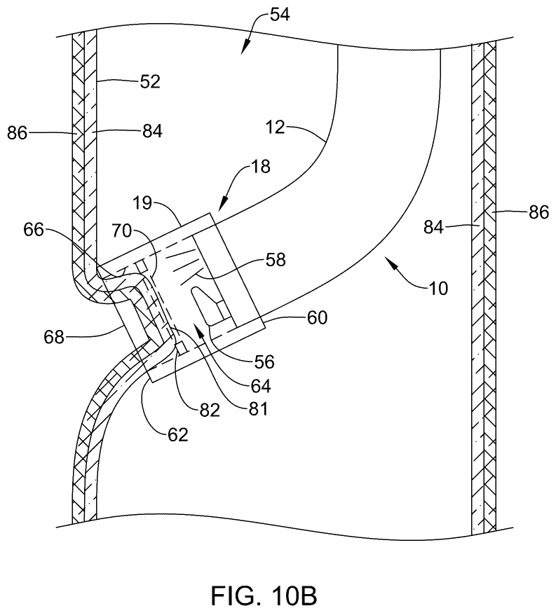

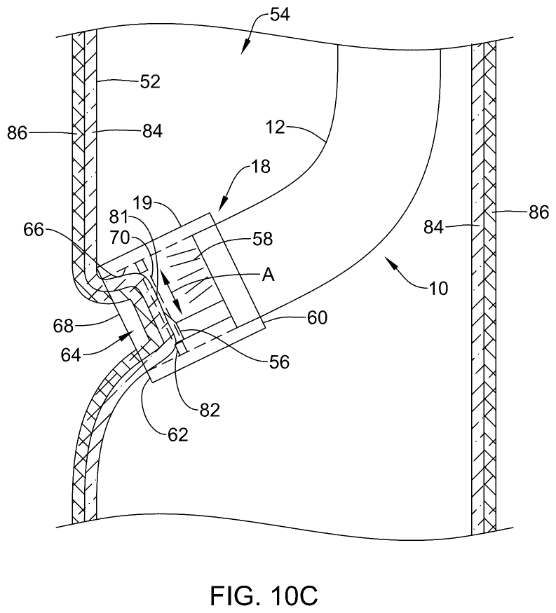

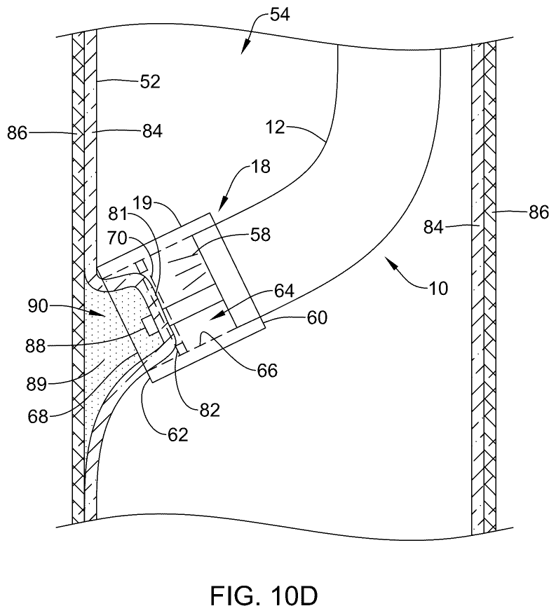

FIGS. 10A-10D depict a schematic flow of an illustrative technique for using the cap 19 in a third space procedure. In some examples, the third space procedure may include creating an incision through a first tissue to at least a second tissue.

FIG. 10A schematically depicts the distal end 18 of the elongate shaft 12 of the medical scope 10 inserted through the GI tract 54 to a target site along the tissue wall 52, where a mucosal layer of tissue 84 defines a lumen of the GI tract 54 and a submucosal layer of tissue 86 may be located in the tissue wall 52 under the mucosal layer of tissue 84. In some examples, the medical scope 10 may include the cap 19 coupled with the distal end 18 of the elongate shaft 12. As depicted in FIG. 10A, the distal end 62 of the cap 19 may engage the tissue wall 52 and may be at a non-zero angle (at a non-flush or non-parallel position and/or orientation) with respect to the tissue wall 52 due to the tight space of the GI tract 54, the rigid nature of the cap 19, and/or other suitable factors.

Once at a target site, a suction force or negative pressure 58 may be applied to and/or through the lumen 64 in the cap 19 to the tissue wall 52 (e.g., from a lumen in the elongate shaft 12 or a tube extending through a lumen in the elongate shaft 12) to draw the tissue wall 52 through the opening 68 and into the lumen 64. As the tissue wall 52 enters the lumen 64, the mucosal layer of tissue 84 of the tissue wall 52 may engage the cap 19 at or proximate the distal end 62 and at or proximate the distal surface 82 of the protrusion 70 to create the flat target surface 81 at which an incision is to be made. In some examples, the distal end 62 of the cap 19 may engage the mucosal layer of tissue 84 prior to initiating the suction force or negative pressure 58, but this is not required. As depicted in FIG. 10C, the protrusion 70 may be proximally inset from the distal end 62 of the cap 19. The cutting tool 56 may be delivered to or otherwise positioned at and/or in the lumen 64 (e.g., via a working channel or other suitable channel of the elongate shaft 12) prior to, during, or after creating the flat target surface 81, as desired.

Once the flat target surface 81 is formed, the cutting tool 56 may engage the flat target surface 81 at or proximate an axial location along the cap 19 at which the protrusion 70 is located (e.g., at a location between two portions of the distal surface 82 of the protrusion 70 and/or at one or more other suitable locations) and create an incision in the tissue wall 52 at the flat target surface 81, as depicted in FIG. 10C. In some examples, the cutting tool 56 may advance and/or retract in the directions of arrow A, along the flat target surface 81 to create an incision of a predictable depth and length. In one example, the incision may be to a depth in the tissue wall 52 at which the submucosal tissue layer starts, but other suitable depts are contemplated.

After an incision of a suitable depth and length is made, the cutting tool 56 may be removed from the lumen 64 and a fluid delivery tool 88 may be inserted into the lumen 64, through the incision, and into the tissue wall 52, as depicted in FIG. 10D. Once a distal end of the fluid delivery tool 88 is inserted into the tissue wall 52, a fluid 89 (e.g., a gas and/or a liquid) may be dispensed from a distal end of the fluid delivery tool 88 to apply pressure between the mucosal tissue layer 84 and the submucosal tissue layer 86 to separate the mucosal tissue layer 84 from the submucosal tissue layer 86 and create a space 90 therebetween. The space between the mucosal tissue layer 84 and the submucosal tissue layer 86 may be used to complete one or more third space procedures, as desired.

The fluid may be any suitable type of fluid. Example suitable types of fluids include, but are not limited to, liquids, gasses, combinations of liquids and gasses, saline solutions, sodium hyaluronate, glycerol solutions, methylcellulose solutions, and/or other suitable fluids.

Although the fluid delivery tool 88 is discussed as delivering the fluid 89, the fluid 89 may be delivered to the tissue wall 52 in one or more other manner. In some examples, the fluid 89 may be delivered to the tissue wall 52 through the cutting tool 56, such that after an incision is made with the cutting tool 56, fluid 89 may be applied to the tissue wall 52 through the cutting tool 56. Alternatively or additionally, the space 90 may be formed in the tissue wall 52 using a spacing tool that is inserted through the incision at the flat target surface 81 and adjusted between the mucosal tissue layer 84 and the submucosal tissue layer 86 to create the space 90.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A cap for a flexible elongate member insertable into a body, comprising:
a proximal end configured to couple with the flexible elongate member;
a distal end defining a first inner diameter;
a lumen extending between the proximal end and the distal end; and
a protrusion located between the proximal end and the distal end and defining a second inner diameter that is smaller than the first inner diameter, and
wherein the protrusion is configured to engage tissue received in the lumen through the distal end.

2. The cap of claim 1, wherein the protrusion is configured to be spaced distally apart from a distal end of the flexible elongate member when the proximal end is coupled with the flexible elongate member.

3. The cap of claim 1, wherein the protrusion extends circumferentially around the lumen.

4. The cap of claim 1, wherein the protrusion defines a non-circular end of the lumen.

5. The cap of claim 1, wherein the protrusion defines a circular end of the lumen.

6. The cap of claim 1, wherein the distal end is made from a substantially transparent material.

7. A device comprising:
a medical scope having a proximal end and a distal end; and
a cap configured to couple with the distal end of the medical scope, the cap comprising:
a proximal end;
a distal end; and
a protrusion having a distal surface configured to engage tissue received through an opening at the distal end.

8. The device of claim 7, wherein the protrusion is spaced distally apart from the distal end of the medical scope when the cap is coupled with the distal end of the medical scope.

9. The device of claim 7, wherein an inner surface of the cap defines a lumen and the lumen has a first diameter at a distal end of the lumen that is greater than a second diameter at the protrusion.

10. The device of claim 9, wherein the protrusion extends circumferentially around the lumen.

11. The device of claim 9, wherein the protrusion defines a non-circular portion of the lumen.

12. The device of claim 9, wherein the protrusion defines a circular portion of the lumen.

13. The device of claim 7, wherein the medical scope has a working channel and an aspiration channel.

14. The device of claim 13, wherein the distal surface of the protrusion and the distal end of the cap are configured to contact tissue when a negative pressure is applied to the aspiration channel.

15. The device of claim 14, further comprising:
a cutting device extending through the working channel, and
wherein the cutting device is configured to cut through a surface of the tissue at a location between two portions of the distal surface engaging the tissue.

16. A method of creating an incision through a first tissue to a second tissue, the method comprising:
engaging a distal end of a cap and a protrusion of the cap with the first tissue, wherein the protrusion is proximally offset from the distal end of the cap; and
cutting through the first tissue to the second tissue at an axial location along the cap proximate an axial location of the protrusion.

17. The method of claim 16, further comprising:
applying a negative pressure to the first tissue through a lumen extending through the cap.

18. The method of claim 16, wherein engaging the distal end of the cap and the protrusion of the cap with first tissue comprises:
engaging the distal end of the cap with the first tissue; and
applying a negative pressure to the first tissue to draw the first tissue into the cap to engage the protrusion.

19. The method of claim 16, wherein a proximal end of the cap is coupled to a distal end of a medical scope and the medical scope has a working channel.

20. The method of claim 19, further comprising:
extending a cutting device through the working channel to a location proximate the axial location of the protrusion.
